SEQUENCE LISTING (99 sequences)

SEQ ID NO: 1 - protein, 6 aa, Artificial Sequence
  Description of Artificial Sequence  peptide ligand
FHALRH

SEQ ID NO: 2 - protein, 6 aa, Artificial Sequence
  Description of Artificial Sequence  peptide ligand
FFSKQN

SEQ ID NO: 3 - protein, 6 aa, Artificial Sequence
  Description of Artificial Sequence  peptide ligand
FPLIKA

SEQ ID NO: 4 - protein, 6 aa, Artificial Sequence
  Description of Artificial Sequence  peptide ligand
FLILDP

SEQ ID NO: 5 - protein, 6 aa, Artificial Sequence
  Description of Artificial Sequence  peptide ligand
FYTPPY

SEQ ID NO: 6 - protein, 6 aa, Artificial Sequence
  Description of Artificial Sequence  peptide ligand
FFYPAX

SEQ ID NO: 7 - protein, 6 aa, Artificial Sequence
  Description of Artificial Sequence  peptide ligand
FLLDKX

SEQ ID NO: 8 - protein, 6 aa, Artificial Sequence
  Description of Artificial Sequence peptide ligand
XRPDKG

SEQ ID NO: 9 - protein, 6 aa, Artificial Sequence
  Description of Artificial Sequence  peptide ligand
FLLFRF

SEQ ID NO: 10 - protein, 6 aa, Artificial Sequence
  Description of Artificial Sequence  peptide ligand
FLLHEE

SEQ ID NO: 11 - protein, 6 aa, Artificial Sequence
  Description of Artificial Sequence  peptide ligand
FLLHPH

SEQ ID NO: 12 - protein, 6 aa, Artificial Sequence
  Description of Artificial Sequence  peptide ligand
FLLHSL

SEQ ID NO: 13 - protein, 6 aa, Artificial Sequence
  Description of Artificial Sequence  peptide ligand
FLLRKF

SEQ ID NO: 14 - protein, 6 aa, Artificial Sequence
  Description of Artificial Sequence peptide ligand
FLLRYS

SEQ ID NO: 15 - protein, 6 aa, Artificial Sequence
  Description of Artificial Sequence  peptide ligand
FLLYRY

SEQ ID NO: 16 - protein, 6 aa, Artificial Sequence
  Description of Artificial Sequence  peptide ligand
FLLQNK

SEQ ID NO: 17 - protein, 6 aa, Artificial Sequence
  Description of Artificial Sequence  peptide ligand
FLLQPF

SEQ ID NO: 18 - protein, 6 aa, Artificial Sequence
  Description of Artificial Sequence  peptide ligand
FLLRFA

SEQ ID NO: 19 - protein, 6 aa, Artificial Sequence
  Description of Artificial Sequence  peptide ligand
FLLRYT

SEQ ID NO: 20 - protein, 6 aa, Artificial Sequence
  Description of Artificial Sequence  peptide ligand
FLLSVI

SEQ ID NO: 21 - protein, 6 aa, Artificial Sequence
  Description of Artificial Sequence  peptide ligand
FLLFRH

SEQ ID NO: 22 - protein, 6 aa, Artificial Sequence
  Description of Artificial Sequence  peptide ligand
FLLKDQ

SEQ ID NO: 23 - protein, 6 aa, Artificial Sequence
  Description of Artificial Sequence  peptide ligand
FLLQYK

SEQ ID NO: 24 - protein, 6 aa, Artificial Sequence
  Description of Artificial Sequence  peptide ligand
FLLTGK

SEQ ID NO: 25 - protein, 6 aa, Artificial Sequence
  Description of Artificial Sequence  peptide ligand
FLLYFT

SEQ ID NO: 26 - protein, 6 aa, Artificial Sequence
  Description of Artificial Sequence  peptide ligand
FLLDLX

SEQ ID NO: 27 - protein, 6 aa, Artificial Sequence
  Description of Artificial Sequence  peptide ligand
FLLYAK

SEQ ID NO: 28 - protein, 6 aa, Artificial Sequence
  Description of Artificial Sequence  peptide ligand
FLLPYK

SEQ ID NO: 29 - protein, 6 aa, Artificial Sequence
  Description of Artificial Sequence  peptide ligand
YRNQFR

SEQ ID NO: 30 - protein, 6 aa, Artificial Sequence
  Description of Artificial Sequence  peptide ligand
AVFNFD

SEQ ID NO: 31 - protein, 5 aa, Artificial Sequence
  Description of Artificial Sequence  peptide ligand
FLLPY

SEQ ID NO: 32 - protein, 4 aa, Artificial Sequence
  Description of Artificial Sequence  peptide ligand
FLLP

SEQ ID NO: 33 - protein, 6 aa, Artificial Sequence
  Description of Artificial Sequence Peptide Ligand
FLLRNV

SEQ ID NO: 34 - protein, 6 aa, Artificial Sequence
  Description of Artificial Sequence Peptide Ligand
FLLNAH

SEQ ID NO: 35 - protein, 6 aa, Artificial Sequence
  Description of Artificial Sequence Peptide Ligand
FLLGPR

SEQ ID NO: 36 - protein, 6 aa, Artificial Sequence
  Description of Artificial Sequence Peptide Ligand
FLLNQE

SEQ ID NO: 37 - protein, 6 aa, Artificial Sequence
  Description of Artificial Sequence Peptide Ligand
FLLFQP

SEQ ID NO: 38 - protein, 6 aa, Artificial Sequence
  Description of Artificial Sequence Peptide Ligand
FLLEVY

SEQ ID NO: 39 - protein, 6 aa, Artificial Sequence
  Description of Artificial Sequence Peptide Ligand
FLLRGS

SEQ ID NO: 40 - protein, 6 aa, Artificial Sequence
  Description of Artificial Sequence Peptide Ligand
FLLIDA

SEQ ID NO: 41 - protein, 6 aa, Artificial Sequence
  Description of Artificial Sequence Peptide Ligand
FLLQSA

SEQ ID NO: 42 - protein, 6 aa, Artificial Sequence
  Description of Artificial Sequence Peptide Ligand
FLLKEI

SEQ ID NO: 43 - protein, 6 aa, Artificial Sequence
  Description of Artificial Sequence Peptide Ligand
FLLPFK

SEQ ID NO: 44 - protein, 6 aa, Artificial Sequence
  Description of Artificial Sequence Peptide Ligand
FLLAPN

SEQ ID NO: 45 - protein, 6 aa, Artificial Sequence
  Description of Artificial Sequence Peptide Ligand
FLLYSA

SEQ ID NO: 46 - protein, 6 aa, Artificial Sequence
  Description of Artificial Sequence Peptide Ligand
FLLLNS

SEQ ID NO: 47 - protein, 6 aa, Artificial Sequence
  Description of Artificial Sequence Peptide Ligand
FLLYRR

SEQ ID NO: 48 - protein, 6 aa, Artificial Sequence
  Description of Artificial Sequence Peptide Ligand
FLLKSV

SEQ ID NO: 49 - protein, 6 aa, Artificial Sequence
  Description of Artificial Sequence Peptide Ligand
FLLLRN

SEQ ID NO: 50 - protein, 6 aa, Artificial Sequence
  Description of Artificial Sequence Peptide Ligand
FLLRGH

SEQ ID NO: 51 - protein, 6 aa, Artificial Sequence
  Description of Artificial Sequence Peptide Ligand
AQTFHD

SEQ ID NO: 52 - protein, 6 aa, Artificial Sequence
  Description of Artificial Sequence Peptide Ligand
RDYDTD

SEQ ID NO: 53 - protein, 6 aa, Artificial Sequence
  Description of Artificial Sequence Peptide Ligand
LKRIEY

SEQ ID NO: 54 - protein, 6 aa, Artificial Sequence
  Description of Artificial Sequence Peptide Ligand
SDLRRL

SEQ ID NO: 55 - protein, 6 aa, Artificial Sequence
  Description of Artificial Sequence Peptide Ligand
APPRTV

SEQ ID NO: 56 - protein, 6 aa, Artificial Sequence
  Description of Artificial Sequence Peptide Ligand
VLYTNN

SEQ ID NO: 57 - protein, 6 aa, Artificial Sequence
  Description of Artificial Sequence Peptide Ligand
NFZQNT

SEQ ID NO: 58 - protein, 6 aa, Artificial Sequence
  Description of Artificial Sequence Peptide Ligand
SKNNAA

SEQ ID NO: 59 - protein, 6 aa, Artificial Sequence
  Description of Artificial Sequence Peptide Ligand
XLLDIR

SEQ ID NO: 60 - protein, 6 aa, Artificial Sequence
  Description of Artificial Sequence Peptide Ligand
XLLKFP

SEQ ID NO: 61 - protein, 6 aa, Artificial Sequence
  Description of Artificial Sequence Peptide Ligand
XLLKQI

SEQ ID NO: 62 - protein, 6 aa, Artificial Sequence
  Description of Artificial Sequence Peptide Ligand
XLLPLK

SEQ ID NO: 63 - protein, 6 aa, Artificial Sequence
  Description of Artificial Sequence Peptide Ligand
XLLQAY

SEQ ID NO: 64 - protein, 6 aa, Artificial Sequence
  Description of Artificial Sequence Peptide Ligand
XLLQHY

SEQ ID NO: 65 - protein, 6 aa, Artificial Sequence
  Description of Artificial Sequence Peptide Ligand
XLLRFT

SEQ ID NO: 66 - protein, 6 aa, Artificial Sequence
  Description of Artificial Sequence Peptide Ligand
XLLYGK

SEQ ID NO: 67 - protein, 6 aa, Artificial Sequence
  Description of Artificial Sequence Peptide Ligand
XLLATI

SEQ ID NO: 68 - protein, 6 aa, Artificial Sequence
  Description of Artificial Sequence Peptide Ligand
XLLDSQ

SEQ ID NO: 69 - protein, 6 aa, Artificial Sequence
  Description of Artificial Sequence Peptide Ligand
XLLEIK

SEQ ID NO: 70 - protein, 6 aa, Artificial Sequence
  Description of Artificial Sequence Peptide Ligand
XLLHPI

SEQ ID NO: 71 - protein, 6 aa, Artificial Sequence
  Description of Artificial Sequence Peptide Ligand
XLLVLP

SEQ ID NO: 72 - protein, 6 aa, Artificial Sequence
  Description of Artificial Sequence Peptide Ligand
XLLAEY

SEQ ID NO: 73 - protein, 6 aa, Artificial Sequence
  Description of Artificial Sequence Peptide Ligand
XLLAFS

SEQ ID NO: 74 - protein, 6 aa, Artificial Sequence
  Description of Artificial Sequence Peptide Ligand
XLLAGV

SEQ ID NO: 75 - protein, 6 aa, Artificial Sequence
  Description of Artificial Sequence Peptide Ligand
XLLHHI

SEQ ID NO: 76 - protein, 6 aa, Artificial Sequence
  Description of Artificial Sequence Peptide Ligand
XLLKGY

SEQ ID NO: 77 - protein, 6 aa, Artificial Sequence
  Description of Artificial Sequence Peptide Ligand
XLLLPK

SEQ ID NO: 78 - protein, 6 aa, Artificial Sequence
  Description of Artificial Sequence Peptide Ligand
XLLPAK

SEQ ID NO: 79 - protein, 6 aa, Artificial Sequence
  Description of Artificial Sequence Peptide Ligand
XLLPFL

SEQ ID NO: 80 - protein, 6 aa, Artificial Sequence
  Description of Artificial Sequence Peptide Ligand
XLLPPR

SEQ ID NO: 81 - protein, 6 aa, Artificial Sequence
  Description of Artificial Sequence Peptide Ligand
XLLPYK

SEQ ID NO: 82 - protein, 6 aa, Artificial Sequence
  Description of Artificial Sequence Peptide Ligand
XLLTSY

SEQ ID NO: 83 - protein, 6 aa, Artificial Sequence
  Description of Artificial Sequence Peptide Ligand
XLLDKX

SEQ ID NO: 84 - protein, 6 aa, Artificial Sequence
  Description of Artificial Sequence Peptide Ligand
XAAFIN

SEQ ID NO: 85 - protein, 6 aa, Artificial Sequence
  Description of Artificial Sequence Peptide Ligand
XLKLFP

SEQ ID NO: 86 - protein, 6 aa, Artificial Sequence
  Description of Artificial Sequence Peptide Ligand
XPNGGI

SEQ ID NO: 87 - protein, 6 aa, Artificial Sequence
  Description of Artificial Sequence Peptide Ligand
XVEVKF

SEQ ID NO: 88 - protein, 6 aa, Artificial Sequence
  Description of Artificial Sequence Peptide Ligand
XFFTFK

SEQ ID NO: 89 - protein, 6 aa, Artificial Sequence
  Description of Artificial Sequence Peptide Ligand
XLLDLX

SEQ ID NO: 90 - protein, 6 aa, Artificial Sequence
  Description of Artificial Sequence Peptide Ligand
XYYLNV

SEQ ID NO: 91 - protein, 6 aa, Artificial Sequence
  Description of Artificial Sequence Peptide Ligand
XAATEH

SEQ ID NO: 92 - protein, 6 aa, Artificial Sequence
  Description of Artificial Sequence Peptide Ligand
XHHPQT

SEQ ID NO: 93 - protein, 6 aa, Artificial Sequence
  Description of Artificial Sequence Peptide Ligand
XSHLLS

SEQ ID NO: 94 - protein, 6 aa, Artificial Sequence
  Description of Artificial Sequence Peptide Ligand
XLHETL

SEQ ID NO: 95 - protein, 6 aa, Artificial Sequence
  Description of Artificial Sequence Peptide Ligand
XAGQGQ

SEQ ID NO: 96 - protein, 6 aa, Artificial Sequence
  Description of Artificial Sequence Peptide Ligand
XDFLKR

SEQ ID NO: 97 - protein, 6 aa, Artificial Sequence
  Description of Artificial Sequence Peptide Ligand
XFVRFX

SEQ ID NO: 98 - protein, 6 aa, Artificial Sequence
  Description of Artificial Sequence Peptide Ligand
XAKVSP

SEQ ID NO: 99 - protein, 6 aa, Artificial Sequence
  Description of Artificial Sequence Peptide Ligand
XYHVYF

(19) United States
(12) Patent Application Publication
(10) Pub. No.: US 2002/0155106 A1
(43) Pub. Date: Oct. 24, 2002

(54) Method of identifying a ligand for a target molecule

(76) Inventor: David J. Hammond, Cortland Manor, NY (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 09/727,963

(22) Filed: Dec. 1, 2000

(51) Int. Cl. C12Q 1/70; A61K 39/00; C07K 5/00; C07K 16/00; A61K 38/00; G01N 33/546; G01N 33/53; C07K 7/00; A61K 38/04; A61K 39/395; C07K 17/00

(52) U.S. Cl. 424/140.1; 435/5; 435/7.1; 530/329; 530/328; 530/327; 530/326; 436/534

(57) ABSTRACT

Ligands that interact with a target, such as one present on a virus, can be more easily identified if false positive interactions (either specific or non-specific) are differentiated from the target-specific interaction. An improved method for screening a library of surface-immobilized ligands which bind to a target is presented. The method can be used for multiple screenings of the same surface-immobilized library for a number of different ligands.

### FIELD OF THE INVENTION

This invention is related generally to a method for identifying ligands, such as polypeptide, peptides and polynucleotides, that bind to targets.

### BACKGROUND OF THE INVENTION

Many biological processes require binding of one macromolecule to a second molecule, which is often referred to as a ligand of the first molecule. Interactions between target molecules and ligands can be important in such biological processes as immune recognition, cell signaling and communication, transcription and translation, intracellular signaling, and enzymatic catalysis. As a result, there is considerable interest in identifying ligands for given molecules. It is also of interest to identify agonists or antagonists of such ligands.

Libraries of polymeric biological or synthetic test compounds are often used to identify ligands for biologically active molecules. Currently, high throughput screening technologies are coupled with the synthesis of compound libraries (obtained through either molecular biological or combinatorial chemistry techniques and other high throughput synthesis methods) to identify rapidly and optimize ligand molecules for biological targets.

Because many biological ligands are proteins or peptides, libraries of putative ligands typically involve peptide design and synthesis. Other libraries can include polymeric compounds (e.g., peptide-like oligomeric compounds) and small organic (non-oligomeric) compounds.

Current methods for screening libraries for ligands for a given target can be hampered by high background levels. These background levels can be associated with binding of agents other than the target to one or more ligands in the library. These agents may be present along with the target in the screening solution used to probe the ligand library.

### SUMMARY OF THE INVENTION

The invention is based in part on the discovery of a rapid, sensitive, and reusable method for screening ligand libraries. The method distinguishes non-specific binding of ligands to agents in the screening solution from specific binding between a ligand and target. A ligand library can be screened for binding to a target, and a profile of ligands specific for the target identified. The process can then be repeated, if desired, with one or more additional targets.

The method includes incubating a plurality of immobilized ligands with one or more solutions that are substantially free of the target. The solution includes one or more agents that can show undesired binding to one or more ligands in the library. The solution or solutions are incubated under conditions that allow for formation of stable complexes between the ligands and agents. Some ligand-agent complexes are detected with a probe molecule having an affinity for the target. The probe molecules bound to the ligand-agent complexes are detected by identifying a signal associated with the complexes.

The ligands are then incubated with a second solution that includes the target and at least one of the solutions used previously. The second solution is also incubated under conditions that allow for formation of stable complexes between the target and the ligands, and for formation of stable complexes between the agents and the ligands. These complexes are then contacted with the probe molecule, and probe molecules bound to ligand-target complexes and probe molecules bound to the ligand-agent complexes are identified.

The signal associated with ligand-probe complexes in the second solution is compared with the transient signal associated with ligand-probe complexes in the first solution. A signal preferentially associated with the second solution compared to the first solution indicates a ligand-probe molecule complex containing a ligand specific for the target.

At least one of the signals used in the screening methods is a transient signal. In preferred embodiments, two or more, or all of the signals used in the methods are transient signals.

Also within the invention is a peptide identified using the herein disclosed screening methods. In one aspect, the invention provides a peptide less than 20 amino acids in length and that includes a porcine parvovirus (PPV)-binding domain, a hepatitis A virus (HAV)-binding domain, or a prion-binding domain. In some embodiments, the peptide is less than 18, 16, 15, 14, or 13 amino acids in length.

In some embodiments, the HAV-binding domain binds to HAV in the presence of fibrinogen. Examples of peptide sequences that include an HAV-binding domain include, e.g., FLLFRF (SEQ ID NO: 9); FLLHEE (SEQ ID NO: 10); FLLHPH (SEQ ID NO: 11); FLLHSL (SEQ ID NO: 12); FLLRKF (SEQ ID NO: 13); FLLRYS (SEQ ID NO: 14); FLLYRY (SEQ ID NO: 15); (F)LLDIR; (F)LLKFP; (F)LLKQI; (F)LLPLK; (F)LLQAY; (F)LLQHY; (F)LLRFT; (F)LLYGK; (F)LLATI; (F)LLDSQ; (F)LLEIK; (F)LLHPI; FLLFRH (SEQ ID NO: 21); FLLKDQ (SEQ ID NO: 22); FLLQYK (SEQ ID NO: 23); FLLTGK (SEQ ID NO: 24); FLLYFT (SEQ ID NO: 25) and (F)LLVLP. An amino indicated in parentheses herein represents a D-Amino Acid.

In some embodiments, the PPV-binding domain binds to PPV in the presence of fibrinogen. Examples of peptide sequences that include a PPV-binding domain include, e.g., (F)LLAEY; (F)LLAFS; (F)LLAGV; (F)LLHHI; (F)LLKGY; (F)LLLPK; (F)LLPAK; (F)LLPFL; (F)LLPPR; (F)LLPYK; FLLQNK (SEQ ID NO: 16); FLLQPF (SEQ ID NO: 17); FLLRFA (SEQ ID NO: 18); FLLRYT (SEQ ID NO: 19); and FLLSVI (SEQ ID NO: 20).

In some embodiments, the HAV-binding domain binds to HAV in the presence of fibrinogen. Example of such peptides that include an HAV-binding domain, e.g, FLLPYK (SEQ ID NO: 28); (F)LLHPI; (F)LLTSY; FLLDLX (SEQ ID NO: 26); (F)LLDKX; and FLLYAK (SEQ ID NO: 27).

In some embodiments, the HAV-binding domain binds PRV. Examples of such HAV-binding domains include, e.g., FHALRH (SEQ ID NO: 1); FFSKQN (SEQ ID NO: 2); (F)AAFIN; (F)LLTSY; (F)LKLFP; (F)PNGGI; (F)VEVKF; FPLIKA (SEQ ID NO: 3); (F)FFTFK; (F)LLDLX; (F)YYLNV; FLILDP (SEQ ID NO: 4); FYTPPY (SEQ ID NO: 5); FFYPAX (SEQ ID NO: 6); FLLDKX (SEQ ID NO: 7) and FLLYAK (SEQ ID NO: 8).

In some embodiments, the peptide includes a sequence that binds to a prion protein, i.e., a prion protein-binding sequence. Examples of proteins including a prion-binding sequence include, e.g., (R)AATEH; (H)HHPQT; (V)SHLLS; (T)LHETL; (V)AGQGQ; (S)DFLKR; (V)FVRFX; (V)AKVSP; (R)YHVYF; (E)RPDKG; YRNQFR (SEQ ID NO: 29); and AVFNFD (SEQ ID NO: 30).

In further embodiments, the peptides include the polypeptide sequence FHALRH (SEQ ID NO: 1); FFSKQN (SEQ ID NO: 2); (F)AAFIN; (F)LLTSY; (F)LKLFP; (F)PNGGI; (F)VEVKF; FPLIKA (SEQ ID NO: 3); (F)FFTFK; (F)LLDLX; (F)YYLNV; FLILDP (SEQ ID NO: 4); FYTPPY (SEQ ID NO: 5); FFYPAX (SEQ ID NO: 6); FLLDKX (SEQ ID NO: 7); FLLYAK (SEQ ID NO: 8); FLLPYK (SEQ ID NO: 28); (F)LLHPI; (R)AATEH; (H)HHPQT; (V)SHLLS; (T)LHETL; (V)AGQGQ; (S)DFLKR; (V)FVRFX; (V)AKVSP; (R)YHVYF; (E)RPDKG; YRNQFR (SEQ ID NO: 29); AVFNFD (SEQ ID NO: 30); FLLFRF (SEQ ID NO: 9); FLLHEE (SEQ ID NO: 10); FLLHPH (SEQ ID NO: 11); FLLHSL (SEQ ID NO: 12); FLLRKF (SEQ ID NO: 13); FLLRYS (SEQ ID NO: 14); FLLYRY (SEQ ID NO: 15); (F)LLDIR; (F)LLKFP; (F)LLKQI; (F)LLPLK; (F)LLQAY; (F)LLQHY; (F)LLRFT; (F)LLYGK; (F)LLATI; (F)LLDSQ; (F)LLEIK; (F)LLHPI; FLLFRH (SEQ ID NO: 21); FLLKDQ (SEQ ID NO: 22); FLLQYK (SEQ ID NO: 23); FLLTGK (SEQ ID NO: 24); FLLYFT (SEQ ID NO: 25); (F)LLVLP; (F)LLAEY; (F)LLAFS; (F)LLAGV; (F)LLHHI; (F)LLKGY; (F)LLLPK; (F)LLPAK; (F)LLPFL; (F)LLPPR; (F)LLPYK; FLLQNK (SEQ ID NO: 16); FLLQPF (SEQ ID NO: 17); FLLRFA (SEQ ID NO: 18); FLLRYT (SEQ ID NO: 19); FLLSVI (SEQ ID NO: 20); (F)LLTSY; FLLDLX (SEQ ID NO: 26); (F)LLDKX; or FLLYAK (SEQ ID NO: 27).

In a further aspect, the invention provides a peptide less than 20 amino acids in length and including a sequence that includes an encephalomyocarditis (EMCV)-binding domain, a simian virus 40 (SV40)-binding domain, a poliovirus-binding domain, a bovine viral bovine viral diarrhea virus (BVDV)-binding domain, or an alpha-1-proteinase inhibitor (API) binding domain.

In some embodiments, the peptide includes an EMCV-binding domain. Examples of peptide sequences with EMCV-binding domains include, e.g., FLLRNV (SEQ ID NO: 33), FLLNAH (SEQ ID NO: 34), FLLGPR (SEQ ID NO: 35), and FLLNQE (SEQ ID NO: 36).

In some embodiments, the peptide includes an SV40-binding domain. Examples of peptide sequences with SV-40 binding domains include, e.g., FLLFQP (SEQ ID NO: 37), FLLEVY (SEQ ID NO: 38), and FLLRGS (SEQ ID NO: 39).

In some embodiments, the peptide includes a poliovirus-binding domain. Examples of peptides with poliovirus-binding domains include, e.g., FLLIDA (SEQ ID NO: 40), FLLQSA (SEQ ID NO: 41), FLLKEI (SEQ ID NO: 42), FLLPFK (SEQ ID NO: 43), FLLAPN (SEQ ID NO: 44), FLLYSA (SEQ ID NO: 45), FLLLNS (SEQ ID NO: 46), FLLYRR (SEQ ID NO: 47), and FLLKSV (SEQ ID NO: 48).

In some embodiments, the peptide includes a BVDV-binding domain. Examples of peptides with BVDV-binding domains include, e.g., FLLLRN (SEQ ID NO: 49), and FLLRGH (SEQ ID NO: 50).

In some embodiments, the peptide includes an API-binding domain. Examples of peptides with API-binding domains include, e.g., AQTFHD (SEQ ID NO: 51), RDYDTD (SEQ ID NO: 52), LKRIEY (SEQ ID NO: 53), SDLRRL (SEQ ID NO: 54), APPRTV (SEQ ID NO: 55), VLYTNN (SEQ ID NO: 56), NFZQNT (SEQ ID NO: 57), and SKNNAA (SEQ ID NO: 58).

In a further aspect, the invention provides a composition that includes a ligand binding peptide described herein. For example, the composition can include a peptide less than 20 amino acids in length and that includes a sequence that includes a PPV-binding domain, an HAV-binding domain, or a prion-binding domain.

In some embodiments, the peptide is coupled to a solid support. The solid support can be, e.g., a resin.

In another aspect, the invention includes a method of removing a target from a biological fluid by contacting the biological fluid with a composition including a ligand binding peptide described herein. The composition is contacted with the target under conditions sufficient to cause specific binding of the target to the peptide in the composition.

In some embodiments, the target is, e.g., HAV, PPV, PRV, prion protein, HIV, EMCV, BVDV, human parvovirus B19 (B19) or SV40. In other embodiments, the target is, e.g., EMCV, SV40, BVDV, API, or poliovirus.

The biological fluid can be, e.g., blood, plasma, serum, cerebrospinal fluid, urine, saliva, milk, ductal fluid, tears, or semen.

In a further aspect, the invention provides a method for identifying a ligand for a target. The method includes (a) incubating a plurality of immobilized ligands with a first solution substantially free of the target and that includes one or more agents under conditions which allow for formation of stable complexes between the ligands and agents; (b) contacting the ligand-agent complexes with a probe molecule having an affinity for the target; (c) identifying probe molecules bound to the ligand-agent complexes by identifying a transient signal associated with the complexes; (d) incubating the ligands with a second solution that includes the target and the first solution under conditions allowing for formation of stable complexes between the target and ligands and for formation of stable complexes between the agents and the ligands; (e) contacting the ligand-target complexes and ligand-agent complexes with the probe molecule; (f) identifying probe molecules bound to ligand-target complexes and probe molecules bound to the ligand-agent complexes in the second solution by detecting the transient signal; and (g) comparing the transient signal associated with ligand-probe complexes in the second solution with the transient signal associated with ligand-probe complexes in the first solution. A signal preferentially associated with the second solution to the first solution indicates a ligand-probe molecule complex that includes a ligand specific for the target.

In some embodiments, the plurality of immobilized ligands is immobilized on individual supports, thereby creating a population of supports. The supports can be, e.g., beads. For example, the plurality of immobilized ligands comprises a combinatorial library of ligand-containing beads. In some embodiments, the population of supports is immobilized on a surface.

In some embodiments, the surface is planar. In some embodiments, the surface is transparent, e.g., the surface can be a planar transparent surface.

In some aspects of the invention, the population of supports is embedded in an adhesive. The adhesive can be, e.g., an elastomeric sealant, such as a vinyl adhesive caulking material.

In some embodiments, the ligand molecules are polymeric. For example, the ligand molecules can be, e.g., peptides, peptidomimetics, small organic molecules, nucleic acids, and carbohydrates.

In some embodiments, the probe molecule is an antibody.

In some embodiments, the detectable transient signal is chemiluminescence. In certain aspects, the chemiluminescence can be detected, e.g., by autoradiography.

In some embodiments, the target is a cell. In other embodiments, the target is a virus. Examples of viral targets include, e.g., porcine parvovirus, pseudorabies virus, hepatitis A virus, poliovirus, simian virus 40, encephalomyocarditis virus, bovine viral diarrhea virus.

In still other embodiments, the target is a protein, e.g., an alpha-1-proteinase inhibitor. In other embodiments, the target is a prion, e.g., a scrapie prion.

In some embodiments, the method further includes removing the agent from the ligand-agent complexes after detecting the transient signal associated with the complexes, and before incubating the ligands with the second solution. In certain embodiments, the method further includes determining the identity of ligands specific for the target.

In a still further aspect, the invention provides a method for identifying a ligand for a plurality of targets. The method includes (a) incubating a plurality of immobilized ligands with a first solution substantially free of a first target and that includes one or more agents under conditions which allow for formation of stable complexes between the ligands and agents; (b) contacting the ligand-agent complexes with a probe molecule having an affinity for the first target; (c) identifying probe molecules bound to the ligand-agent complexes by identifying a transient signal associated with the complexes; (d) removing the agent from the ligand-agent complexes; (e) incubating the ligands with a second solution that includes the first target and the first solution under conditions allowing for formation of stable complexes between the first target and ligands and for formation of stable complexes between the agents and the ligands; (f) contacting the ligand-first target complexes and ligand-agent complexes with the probe molecule; (g) identifying probe molecules bound to ligand-first target complexes and probe molecules bound to the ligand-agent complexes in the second solution by detecting the transient signal; (h) comparing the transient signal associated with ligand-probe complexes in the second solution with the transient signal associated with ligand-probe complexes in the first solution, wherein a signal preferentially associated with the second solution to the first solution indicates a ligand-probe molecule complex that includes a ligand specific for the first target; (i) removing the agents from the ligand-agent complexes and the first targets from the ligand-first target complexes, and j) repeating steps (e) through (i) with a third solution that includes a second target.

In some embodiments, the method further includes determining the identity of a ligand specific for first target or second target.

In another aspect, the invention includes a method for identifying a peptide that binds to a virus present in a blood composition. The method includes (a) providing a plurality of immobilized beads that includes a combinatorial peptide library; (b) incubating the peptide library with a blood composition substantially free of the virus and that includes one or more agents, under conditions which allow for formation of stable complexes between the peptides and agents; (c) contacting the complexes with a first antibody having an affinity for the virus under conditions that allow formation of peptide-agent-first antibody complexes; (d) identifying a transient signal associated with the peptide-agent complexes; (e) contacting the peptides with a second blood composition that includes the virus molecule and the first blood composition under conditions allowing for formation of stable peptide-virus complexes and peptide-agent complexes; (g) identifying a transient signal associated with the peptide-virus complexes and peptide-agent complexes; and (h) comparing the transient signal associated with peptide-virus complexes and the transient signal associated with peptide-agent complexes with the transient signal associated with peptide-agent complexes in the first blood composition, wherein a transient signal preferentially associated with the second blood composition to the first blood composition indicates a peptide-virus complex that includes a peptide specific for the virus.

In some embodiments, the method further includes determining the identity of a ligand specific for the first target or second target. In some aspects, the method includes identifying the sequences of the peptides.

In some embodiments, the peptide library is immobilized on a transparent film using a vinyl caulking adhesive.

In some embodiments, the method further includes repeating steps (e) through (i) with a third blood composition that includes a second target. In some embodiments, the method further includes repeating steps (e) through (i) with a fourth blood composition that includes a third target.

The invention offers many advantages. For example, it offers the ability to differentiate between binding reactions involving specific ligand-target interactions from those that involve binding of beads (and/or the corresponding ligand) to agents contained in the reaction media.

Another advantage of the invention is that the ligand is not consumed in the screening process. In addition, ligand binding is detected using a transient signal that is associated with a bound probe molecule. Thus, multiple probe molecules can be applied successively to the same immobilized library of ligands. A series of profiles can be generated and the results directly compared to each other, e.g., by comparing the binding profiles for ligand with a number of different targets.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention describes a method of identifying a ligand for a target. A target can be any molecule or complex of interest. It can thus include, e.g., a polypeptide, lipopolysaccharide, a polysaccharide or other molecule of biological interest. It can in addition include a macromolecular structure, such as a virus or cell. The method can optionally be referred to as QuASAR™ screening: “Quick Assay for Selecting Affinity Resins”.

The method uses a subtractive analytical technique select for members of a ligand library that specifically bind to the target. In general, a two-step screening procedure is used. First, an immobilized library of ligands is incubated with a screening solution devoid of a target. The screening solution may contain one or more agents which bind to one or more ligands in the library to form an agent-ligand complex. The ligand-agent complex may then be detected by the detection system used to identify specific ligand-target complexes. Unless identified, agent-ligand complexes can obfuscate identification of ligands that are specific for target.

The screening solution will typically be the solution in which the target will be incubated with the screening library. For example, when the target is a growth factor secreted into a cell culture medium, the screening solution can be the cell culture medium lacking the secreted growth factor. Other screening solutions can be, e.g., a viral growth medium when targets corresponding to viruses are to be tested, or other fluids such as blood compositions when targets in these compositions are to be tested. Another suitable first screening solution is a solution that includes the probe molecule. This screen will identify those library members which bind to the probe molecule in the absence of an agent or target.

If desired, multiple screening solutions lacking a target can be used, e.g., one screening solution may include the probe molecule in the absence of agents present in the screening solution and another prescreening solution may include a cellular extract lacking the target. Thus, for screens for targets raised against a prion protein, suitable presecreening solutions include a first solution containing an antibody for a prion protein and a second solution containing a “blank” or “control” brain extract in which the infectious prion protein is known or suspected of being absent.

Next, a probe molecule is added to identify ligand-agent complexes. In general any probe molecule can be used, so long as it recognizes the target. Probe molecules can be, e.g., an antibody to a virus or viral protein, when the virus or viral protein is used as a probe, or an antibody to a protein or growth factor when the protein or growth factor is used as the target.

Probe molecules which detect agents bound to one or more ligands in the library are identified by generating a signal that is associated with the bound probe molecule. In some embodiments, the signal is recorded on X-ray (autoradiography) film, and the pattern of exposures on the film is referred to as a star map. The exposed portions of the film indicate regions in a ligand library that have bound to the probe molecule.

After probe molecules recognizing agent-ligand complexes are identified, the immobilized ligands are probed with a second screening solution. As noted above, the second solution typically will include all the components of the first solution, as well as the target. The second screening solution and ligand library are incubated under conditions which allow for the formation of ligand-target complexes, as well as complexes between one or more agents in the screening solution and ligands.

Following incubation with the second solution molecule, the probe molecule is added to detect agents and targets bound to one or more beads in the library. Probe molecules which bind to the target-ligand complex, or to agent-ligand complexes are identified by generating a transient signal that is associated with the bound probe molecule. The transient signal is recorded as in the first screen, to produce a second star map. The map includes signals at the same positions as those observed following incubation with the first screening solution, as well as a new set of signals. It is understood that screening may be performed in the two steps by generating the same type of signal, wherein the various map positions are labeled in the same manner, or, alternatively, the screening may be performed in the two steps by generating a different type of signal, wherein the various map positions are labeled in a different manner

Identifying signals associated with the target requires comparing the signals generated in the first and second steps. The second set of signals typically has a majority of signals in common to the first set and corresponds to signal from beads that bind to the reagents used in probing for ligand as well as signal from ligand-target binding. Subtraction of the first set of signals from the second reveals a set of signals that corresponds to beads that contain ligands that bind specifically to the target.

At least one signal used to identify a probe molecule in the first or second screening steps is a transient signal. By transient is meant a signal whose duration is finite. In some embodiments, the signal lasts 14 days or less. For example the signal may last for seven days, three days, two days, one day, 12 hours, six hours, three hours, one hour, or 30 minutes or less. The transient signal associated with bound agent-ligand complexes or target-agent complexes is typically recorded and stored on a permanent medium. In preferred embodiments, both screening steps are detected using a transient signal.

If desired, the same library of ligands can be screened for binding to a variety of targets. For example, individual beads, each carrying a unique ligand, are immobilized on a thin film of adhesive and probed with a screening solution such as culture medium (“control” or “blank”) in the absence of target. Beads that bind to the agents in the screening solution are identified, as are beads that cross-react to the probe. The screening solution is then removed and, after washing, the probing procedure is repeated with medium containing the target. After signal generation and identification the agents, targets, and probe molecules are removed, thereby making the ligands in the library available again for binding. This procedure is also referred to as regenerating the ligand library. The regenerated library can then be re-screened for binding to different targets. Upon completion of all screens, the beads corresponding to specific signals are removed from the adhesive, and the ligands that bind to one or more targets are characterized. For example, a particular library can be screened many different times for ligands that bind to members of a family of targets, such as a group of proteins thought to have a similar structural motif, or a variety of viruses found in blood. A comparison of the ligands that bind effectively to a number of targets in a particular family provides useful information for the design of therapeutic agents.

The method can additionally be used to identify ligands that bind to one or more targets but which do not bind to a second target or targets. In addition, the method can be used to identify ligands that bind to targets in the presence of a component or components of a biological fluid, such as the blood-associated protein fibrinogen. The components, such as fibrinogen, may compete with target for the ligand.

The screening methods described herein generally refer to an initial screening or screenings with solutions lacking the target, followed by screening with solutions containing the target. However, this temporal sequence is not obligatory. Thus, unless stated otherwise the herein described methods encompass steps in which a ligand library is initially screened with a solution or solutions including one or more targets, followed by screening with a solution or solutions lacking the targets.

The method described herein can be used in a variety of applications. Libraries of ligand molecules can be screened, for example, for their ability to bind to target (“receptor”) molecules. These targets include, for example, proteins, nucleic acids, carbohydrates, small molecules, or cells. In general, any type of cell can be used in the assay. Thus, cells can include prokaryotic or eukaryotic cells. Examples of prokaryotic cells include, e.g., gram negative bacteria (e.g., Yersinia spp.) or gram positive bacteria (e.g., Staphylococcus or Streptococcus spp.) Eukaryotic cells can include single-celled organisms, e.g., protozoan or fungal cells (e.g., yeast such as Candida spp.) or cells derived from metazoan organisms. For example, in some embodiments, cells can be associated with various components of mammalian blood. Thus, cells can include, e.g., platelets, erythrocytes, lymphocytes.

The term “target” refers to the entity for which a binding ligand (such as a nucleic acid, peptide, peptoid, or other compound capable of binding thereto) is desired. By identifying the ligand de novo, one need not know the sequence or structure of the target or the identity or structure of its natural binding partner. Indeed, for many “receptor” molecules, a binding partner has not been identified.

As used herein, a library means at least two, (e.g., 5; 10; 50; 100; 200; 500; 1,000; 2,500; 5,000; 10,000; 25,000; or more) molecular entities with different sequences. Libraries can include polymeric compounds such as nucleic acids, carbohydrates, or peptides. In the case of peptide libraries, the amino acid building blocks can be the 20 genetically encoded L-amino acids, D-amino acids, synthetic amino acids, amino acids with side chain modifications such as sulfate groups, phosphate groups, carbohydrate moieties, etc. As used herein, the D isomers of the amino acids are represented in parentheses, e.g., (F) denotes the D-isomer of phenylalanine. A random peptide library may include a mixture of peptides ranging in length from 2-100 amino acids or more in length, but are typically about 5-15 amino acids in length. The term “random” indicates only the most typical preparation of the library, and does not require that the composition be unknown. Thus, one may prepare a mixture of precisely known composition if desired. The libraries can also include non-oligomeric ligands, e.g., small non-oligomeric organic compounds.

In general, any art-recognized method for constructing a ligand library can be used. The development of synthetic peptide combinatorial libraries on inert surfaces has made available large numbers of distinct peptides for studying ligand-target interactions. Random peptide libraries can be produced by standard organic synthesis of amino acids polymerized on micro beads. Typically, the peptides on any one bead in a library are substantially the same; however, the peptide sequences vary from bead to bead. For example, a mix, divide and couple synthesis method can be used generate unique peptide sequences on polystyrene-based resinous beads, as described in Furka et al., Int. J. Pept. Protein Res. 37: 487-493 (1991); Lam et al., Nature 354: 82-84 (1991). Alternatively, surface-bound, chemically synthesized libraries can now be purchased from commercial vendors. For example, the library can be obtained from Peptides International, Inc. (Louisville, Ky.).

A library of ligands can be affixed to a surface (e.g., bead) using any attachment method which results in a linkage that is stable enough that the relative spatial locations of members of the library can be detected on the surface.

In some embodiments, a library containing peptide ligands is constructed by synthesizing peptides on a TentaGel resin (Peptides International, Inc., Louisville, Ky.). This gelatinous resin is constructed from a backbone of low-crosslinked polystyrene grafted with polyoxyethylene (polyethylene glycol). The typical chain length of polyethylene is 68 ethylene oxide units. At the end of the polyethylene is a reactive group (e.g. primary amine or thiol) to which the ligand is attached. For example, the reactive group is the site of attachment of a first protected amino acid that is coupled through its carboxy group. Following coupling, the protected amino group of the first amino acid is deprotected to expose a new amino terminus. This new amino terminus functions as a site for attachment of the next protected amino acid. Through cycles of coupling and deprotection the library is grown from the initial reactive group of the TentaGel resin.

In some embodiments, individual beads, each carrying a unique ligand, are immobilized on a flat surface. For example the surface on to which a ligand library is attached can be an inert polymeric sheet. In most preferred embodiments, the surface is a transparency film. An adhesive can be used to immobilize the beads. A preferred adhesive is metal-gray PHENOSEAL™ vinyl adhesive caulk (Gloucester Company, Inc. Franklin, Mass.).

Binding of an agent or target to a ligand can be detected with a probe molecule by employing any art-recognized method. At least one screening step identifies a target using a transient signal associated with the presence of the probe molecule. For example, there are several methods commonly used in the art for detecting the binding of a target to a surface-immobilized ligand.

Any detectable difference between non-bound ligands and ligands bound to targets can be exploited. For example, a probe molecule which recognizes the target can be added to the screened ligand library and allowed to bind. Alternatively (or in addition), a fluorescently labeled target bound to ligands can be detected spectrophotometrically. Radioactively labeled probe molecules may also be used, for example the label can be radioactive iodine. In other embodiments, antibodies to the target may be used as detection molecules. In certain embodiments, the target is not itself labeled, but after the binding reaction, the target is reacted to produce a detectable transient signal (i.e., a transient light emitting signal).

In one embodiment, detection of a binding event involves probing the ligand-target complex with screening solution containing a primary antibody that recognizes the target, followed by a secondary antibody/phosphatase conjugate that binds to the primary antibody. Beads with ligands bound to either antibody or phosphatase are visualized by adding a chemiluminescent substrate (e.g., CDP-Star®, from Tropix, Inc., Bedford Mass.) to detect the presence of phosphatase. Detection of light production is captured on an autoradiography film. The resulting film appears as a ‘star map’, with the intensity of signals on the film reflecting the intensity of light generation from the different beads.

Once a ligand-target complex has been detected, the ligand is identified and further characterized. Identifying the ligand may include re-screening some members in an original library from a region of a ligand library containing putative target-binding ligand. This will typically be performed when the ligand library is plated at a relatively high density.

In one embodiment, peptide ligands that bind to a number of different types of targets are identified. In certain embodiments, the target is a small molecule or a polymer, such as a protein, nucleic acid or carbohydrate. In other embodiments, the target is a large cell, such as an erythrocyte, lymphocyte, or platelet. In still other embodiments, the target is an entire organism, such as a bacterium.

In one aspect, a peptide library is screened for ligands capable of binding to one or more viruses. For example, one suitable viral target is the viral canyon structural motif, which is a deep depression, or canyon, in the viral capsid. See, Rossmann et al., Nature 317: 145-153 (1985); Colonno et al., Proc. Natl. Acad. Sci. (U.S.A.) 85: 5449-5453 (1988); Rossmann et al., Ann. Rev. Biochem. 58: 533-573 (1989). Residues at the floor of this canyon are implicated in IntraCellular Adhesion Molecule-1 (ICAM-1) binding. Thus, peptides in a ligand library can be screened for the ability to bind to a common structure such as a canyon on the surface of viruses and possibly mimic ICAM-1 binding.

Peptide ligands identified using QuASAR™ screening

Also disclosed herein are ligands which bind to various viral or protein targets. Accordingly, the invention also includes peptides that include the amino acid sequences of these target-binding peptides. In some embodiments, the invention includes a peptide less than 20 amino acids in length (e.g., less than 18, 15, 12, 10, or 8 amino acids) and which includes a sequence that binds to a target. The sequence in the peptide that binds to the target is referred to as a “binding domain”. Thus, the peptide may include an amino acid sequence defining a domain that binds to e.g., polio virus, pseudorabies virus (PRV), hepatitis A virus (HAV), porcine parvovirus (PPV), encephalomyocarditis virus (EMCV), human parvovirus B19 (B19), bovine viral diarrhea virus (BVDV), simian virus 40 (SV40),. human immunodeficiency virus (HIV), or a protein target, such as prion protein or alpha-1-proteinase inhibitor (API). In some embodiments, the binding domain binds to a target, e.g., HAV, PRV, or PPV in the presence a constituent of a bodily fluid, e.g., the peptide binds to the target in the presence of the blood-associated protein fibrinogen. Peptides having the amino acid sequences shown in Table 4, samples 1-12 and 15-17 bind HAV targets in the presence of screening solutions which contain plasma or fibrinogen. Peptides having the amino acid sequences shown in Table 4, samples 18-32 bind PPV in the presence of plasma or fibrinogen, while peptides having the amino acid sequences shown in Table 4, samples 33-42 bind HAV but not first screening solutions having whole blood.

Some peptides bind to more than one target. For example some peptides bind to two, three or more targets. For example, some peptides bind both HAV or PPV. Preferably, these peptides showing degenerate binding show little or no affinity for components of endogenous body fluids, such as fibrinogen. Examples of peptides that bind to HAV, PPV and PRV, but not constituents of bodily fluids, are those represented in Table 4, samples 43-46.

Examples of peptides which bind to HAV, PAV and PRV are FHALRH (SEQ ID NO: 1), FFSKQN (SEQ ID NO: 2), (F)AAFIN, (F)LLTSY, (F)LKLFP, (F)PNGGI, (F)VEVKF, FPLIKA (SEQ ID NO: 3), (F)FFTFK, (F)LLDLX, (F)YYLNV, FLILDP (SEQ ID NO: 4), FYTPPY (SEQ ID NO: 5), FFYPAX (SEQ ID NO: 6), FLLDKX (SEQ ID NO: 7) and FLLYAK (SEQ ID NO: 8), where X denotes an amino acid whose identity is ambiguous after sequencing, and X can be any of the 17 amino acids used in the construction of the library.

Peptides which bind prion proteins from hamsters infected with scrapie, but not to brain extracts from healthy litter-mates include those having the amino acid sequences shown in Table 6. Peptides with these sequences also do not bind to a control solution containing a probe molecule, monoclonal antibody FH 11, which recognizes the amino terminal region of a recombinant PrP protein.

Polypeptides that include these sequences are useful for a variety of analytical, preparative, and diagnostic applications. For example, they can be coupled to a solid support, such as a resin and used to bind targets that are present in a solution, e.g., a biological fluid. See, e.g., Doyle, U.S. Pat. No. 5,750,344. Examples of biological fluids include, e.g., blood or blood components, such as plasma or serum. Additional fluids include cerebrospinal fluid, urine, saliva, milk, ductal fluid, tears, or semen.

Binding can reveal the presence of the target in the solution. Alternatively, or in addition, binding can be used to selectively remove the cognate target or targets from the biological fluid. For selective detection and removal of targets from solutions the peptides can be attached to a solid supports, such as a resin. Resins for removing agents from fluids such as blood or blood products are well known in the art and are described in, e.g., Horowitz et al., U.S. Pat. No. 5,541,294; Buettner et al., U.S. Pat. No. 5,723,579; Buettner, U.S. Pat. No. 5,834,318. Among the targets which can be removed are HAV, PPV, PRV, simian virus 40 (SV40), HIV, encephalomyocarditis virus (EMCV), human parvovirus B19 (B19), bovine viral diarrhea virus (BVDV), polio virus and proteins such as prion protein and API. Furthermore, the target can be gram positive or gram negative bacterium.

The following examples are presented in order to more fully illustrate the preferred embodiments of the invention. These examples do not limit the scope of the invention as defined by the appended claims.

### EXAMPLE 1

Identification of peptides that bind to virus using QuASAR™ screening

The QuASAR™ screening method is used to identify peptides that specifically bind to targets found in blood.

A thin film of PHENOSEAL™ adhesive is spread on a transparency film. Two strips of transparent adhesive tape are stuck in parallel to the surface, and the PHENOSEAL™ adhesive is spread between the pieces of tape with a metal tube. The tape is used as a spacer to provide a film of adhesive the thickness of the tape. Approximately 1000 beads (5 mg) from each of 33 sublibraries are placed in water in a thin layer on a plastic wrap. The transparency covered with adhesive is inverted onto the bead solution so that the beads become stuck to the solid surface by means of the adhesive. The transparency/wrap are then inverted with the wrap now on top. The wrap is removed and the beads remain fixed to the PHENOSEAL™ adhesive.

The tops of the beads are left in contact with the air. After the adhesive is allowed to harden for a few hours, the beads are blocked (2 hours to overnight) at 4° C. with a protein solution. Usually, casein is used as a blocking agent. The blocking agent adheres to surfaces of damaged beads to prevent non-specific binding of the target to hydrophobic surfaces. In some experiments, the beads are pre-washed with regeneration buffer (see below) prior to the blocking step.

The surface-immobilized library is next incubated with fresh media (e.g., plasma) for two hours at room temperature. The beads are then washed six times for five minutes each with TS (0.05 M Tris, 0.15 M NaCl, pH 7.4). This wash removes any weakly bound protein from the beads. The beads are typically incubated overnight with primary antibody to the target (e.g., protein or virus) of interest. The wash step is then repeated to remove any weakly bound primary antibody. The beads are incubated with labeled secondary antibody, which is specific for the primary antibody, for about 30 min. In most trials, the secondary antibody is labeled with phosphatase. After incubation with antibody, the beads are washed six times with TS plus 0.05% Tween, and six washes of TS. All washes are 10 min long. Extensive washing protocols are necessary to remove all non-bound phosphatase. After the final TS wash, the beads are washed with 0.05 M Tris pH 9.5 plus 0.005 M MgCl₂ for 10 min. The higher pH is necessary for the activity of bound phosphatase.

When phosphatase-labeled secondary antibody is used, the phosphatase associated with each bead is developed by adding a chemiluminescent substrate (e.g., CDP-Star®, from Tropix, Inc., Bedford Mass.) in 0.05 M Tris pH 9.5, 0.005 M MgCl₂. The substrate is gently applied to the surface of the beads, and the chemiluminescence is detected by autoradiography: the immobilized beads are wrapped in waterproof cling film, and are exposed to autoradiography film.

Beads emitting a chemiluminescent signal produce a pattern of signals, or a “star map”, on the film. The location of the film on the transparency is recorded in some cases by marking with three pinholes. Exposure time is assessed empirically, based on the strength of the signal produced. For the background screen, the film is over-exposed, usually an overnight exposure, to ensure that even beads producing a weak signal are detected. Because of the sensitivity of chemiluminescent assays, a high percentage of beads eventually produce a detectable signal over very extended periods of incubation, e.g. 72 hours or longer. This overexposure facilitates precise alignment of the beads with the pattern on the autoradiography film.

To regenerate the beads for the target screen, the reagents are flushed from the beads typically by washing in 3 M sodium thiosulfate for 15 min; 6 M urea, and 2% acetic acid for three washes each of 15 min. The pH is equilibrated with sufficient washes of 0.15 M citrate, pH 7.4, to bring the pH to about 7. To detect beads containing a peptide that binds specifically to the target, the above procedure of blocking and probing is duplicated except that the target is present in the two hour binding incubation. In some screens, the target is PPV, PRV, or HAV. In others, the target is scrapie protein or API.

A second autoradiogram is produced as described above, and compared with the first, background, autoradiogram. Beads that are detected only in the second screen in the presence of target are identified. This sub-set of beads is usually indicated by placing a red mark on the spots on the over developed autoradiography film that corresponded to the target-specific beads. The film is placed under the transparent support and the beads are lined up with their corresponding spots on the autoradiogram. The film and transparency are taped together to keep the alignment accurate. Beads over red spots are easily identified visually or with a microscope.

Individual positive beads are removed from the adhesive according to the following procedure: the surface of the adhesive is moistened with a saline solution, and the positive beads (as indicated by a red dot) are picked from the adhesive using a syringe needle. They re then placed individually in small tubes and stained with Coomassie blue to facilitate handling. The beads are then loaded into a protein sequencer and the ligands on the positive beads are characterized by Edman degradation.

Examples of ligands identified for various viral targets are shown in Table 1. All beads begin with either (F)LL or FLL at positions 1-3. The results of sequences at positions 4-6 of the positive beads are outlined in Table 1.

TABLE 1

| Sample | SEQ ID | Position 1-3 | Position 4 | Position 5 | Position 6 | Screen |
|---|---|---|---|---|---|---|
| 1 | SEQ ID NO:33 | FLL | R | N | V | EMCV |
| 2 | SEQ ID NO:34 | FLL | N | A | H | |
| 3 | SEQ ID NO:35 | FLL | G | P | R | |
| 4 | SEQ ID NO:36 | FLL | N | Q | E | |
| 5 | SEQ ID NO:37 | FLL | F | Q | P | SV40 |
| 6 | SEQ ID NO:38 | FLL | E | V | Y | |
| 7 | SEQ ID NO:39 | FLL | R | G | S | |
| 8 | SEQ ID NO:40 | FLL | I | D | A | Polio |
| 9 | SEQ ID NO:41 | FLL | Q | S | A | Virus |
| 10 | SEQ ID NO:42 | FLL | K | E | I | |
| 11 | SEQ ID NO:43 | FLL | P | F | K | |
| 12 | SEQ ID NO:44 | FLL | A | P | N | |
| 15 | SEQ ID NO:45 | FLL | Y | S | A | |
| 16 | SEQ ID NO:46 | FLL | L | N | S | |
| 17 | SEQ ID NO:47 | FLL | Y | R | R | |
| 18 | SEQ ID NO:48 | FLL | K | S | V | |
| 19 | SEQ ID NO:49 | FLL | L | R | N | BVDV |
| 20 | SEQ ID NO:50 | FLL | R | G | H | |

Examples of ligands that bind to alpha-1-proteinase inhibitor (API) are shown in Table deficiency of API protein is thought to be involved in premature development of pulmonary emphysema. See, e.g., U.S. Pat. No. 4,496,689.

TABLE 2

| | | Position | Position | Position | Position | Position | Position |
|---|---|---|---|---|---|---|---|
| Sample | SEQ ID | 1 | 2 | 3 | 4 | 5 | 6 |
| 1 | SEQ ID NO:51 | A | Q | T | F | H | D |
| 2 | SEQ ID NO:52 | R | D | Y | D | T | D |
| 3 | SEQ ID NO:53 | L | K | R | I | E | Y |
| 4 | SEQ ID NO:54 | S | D | L | R | R | L |
| 5 | SEQ ID NO:55 | A | P | P | R | T | V |
| 6 | SEQ ID NO:56 | V | L | Y | T | N | N |
| 7 | SEQ ID NO:57 | N | F | Z | Q | N | T |
| 8 | SEQ ID NO:58 | S | K | N | N | A | A |

### EXAMPLE 2

Identification of peptides that bind to multiple viruses using QuASAR™ screening

QuASAR™ screening is used to identify peptide ligands that bind to one or more viruses, even in the presence of fibrinogen, or components of whole blood or serum.

Thirty-three sublibraries are synthesized on TentaGel resin. Each bead has a six amino acid sequence synthesized onto a free amino group. These libraries have a D- or L-amino acid at the N-terminus (position 1). All of the common amino acids are used except cysteine, methionine and tryptophan. The five other positions (positions 2-6) have only L-amino acids. Each sublibrary is individually screened with antibody to PPV followed by chemiluminescent detection as described above. The sublibraries are then re-screened in the presence of PPV as described above. The number of beads specific for PPV binding in each sublibrary is counted. Of the 81 positive signals, 56 are present in the sublibrary containing peptides beginning with D-phenylalanine, (F). Three signals are present in the sublibrary beginning with phenylalanine, F; three in the sublibrary beginning with L-alanine, A; three in the sublibrary beginning with L-proline, P; and three in the sublibrary beginning with D-leucine, (L).

The sublibraries starting with (F) and F are further analyzed by plating them and probing with the viruses PPV, HAV and PRV. Sixteen sequences that bind to all three viruses are obtained and their amino acid sequence is determined. These peptides and their sequences include: FHALRH (SEQ ID NO: 1), FFSKQN (SEQ ID NO: 2), (F)AAFIN, (F)LLTSY, (F)LKLFP, (F)PNGGI, (F)VEVKF, FPLIKA (SEQ ID NO: 3), (F)FFTFK, (F)LLDLX, (F)YYLNV, FLILDP (SEQ ID NO: 4), FYTPPY (SEQ ID NO: 5), FFYPAX (SEQ ID NO: 6), FLLDKX (SEQ ID NO: 7) and FLLYAK (SEQ ID NO: 8), where X denotes an amino acid whose identity is ambiguous after sequencing, and X is any of the 17 amino acids used in the construction of the library.

The sequences do not show a random distribution of amino acids. For example, one amino acid, L, is found in position 2 six times, but not once in position 6. Additionally, four of the sixteen peptides begin with FLL. Statistically 1 in 289 are anticipated to begin with these three amino acids, indicating that this sequence is biased for virus binding.

To further define a peptide sequence which binds to multiple viruses, sublibraries based on the (F)LLXXX FLLXXX sequence motifs are synthesized and screened for binding of PPV, PRV, poliovirus, EMCV, BVDV, SV40 and HAV.

The signal intensity associated with each virus is classified according to whether signals are observed following probing with HAV, PPV, PRV, or media alone. The intensity of signal is considered for each bead based on the range of exposures for that particular plate and is recorded on a +/− scale with [−] being non-detectable and [++++] being the highest intensity in the star map. An example of a bead description table is illustrated as Table 3 below:

TABLE 3

| Bead ID | HAV | PPV | PRV | Media | Media o/n |
|---|---|---|---|---|---|
| 01 | ++++ | + | ++++ | − | Faint |

### EXAMPLE 3

Identification of peptides using QuASAR™ screening that bind to multiple viruses in the presence of components of bodily fluids

Background binding is an important consideration in the screening for ligands that bind specifically to target when the target is present in a solution having one or more components which may compete the ligand for binding to the target. These competing ligands may be found in, e.g., bodily fluids, notably fibrinogen. Experiments targeting receptor binding domains of virus are therefore designed to select against peptides that also bind fibrinogen through the ICAM binding site. The screening procedure is essentially as described in Example 1, with the exception that a screen for beads that bind to virus in the presence of excess fibrinogen is performed, i.e. fibrinogen is an agent in the medium of the first screen.

Using the procedure described in Example 1, a library of hexameric peptides immobilized on beads is screened for HAV, PPV, and PRV in plasma, fibrinogen solution, or whole blood. The results are presented in the table below. Sequences that bind all three viruses are also indicated. All beads begin with either (F)LL or FLL at positions 1-3. The results of sequences at positions 4-6 of the positive beads are outlined in Table 4.

TABLE 4

| Sample | SEQ ID | Position 1-3 | Position 4 | Position 5 | Position 6 | Screen |
|---|---|---|---|---|---|---|
| 1 | SEQ ID NO:9 | FLL | F | R | F | HAV |
| 2 | SEQ ID NO:10 | FLL | H | E | E | (Plasma |
| 3 | SEQ ID NO:11 | FLL | H | P | H | Or |
| 4 | SEQ ID NO:12 | FLL | H | S | L | Fibrinogen) |
| 5 | SEQ ID NO:13 | FLL | R | K | F | |
| 6 | SEQ ID NO:14 | ELL | R | Y | S | |
| 7 | SEQ ID NO:15 | FLL | Y | R | Y | |
| 8 | | (F)LL | D | I | R | |
| 9 | | (F)LL | K | F | P | |
| 10 | | (F)LL | K | Q | I | |
| 11 | | (F)LL | P | L | K | |
| 12 | | (F)LL | Q | A | Y | |
| 15 | | (F)LL | Q | H | Y | |
| 16 | | (F)LL | R | F | T | |
| 17 | | (F)LL | Y | G | K | |
| 18 | | (F)LL | A | E | Y | PPV |
| 19 | | (F)LL | A | F | S | (Plasma |
| 20 | | (F)LL | A | G | V | Or |
| 21 | | (F)LL | H | H | I | Fibrinogen) |
| 22 | | (F)LL | K | G | Y | |
| 23 | | (F)LL | L | P | K | |
| 24 | | (F)LL | P | A | K | |
| 25 | | (F)LL | P | F | L | |
| 26 | | (F)LL | P | P | R | |
| 27 | | (F)LL | P | Y | K | |
| 28 | SEQ ID NO:16 | FLL | Q | N | K | |
| 29 | SEQ ID NO:17 | FLL | Q | P | F | |
| 30 | SEQ ID NO:18 | FLL | R | F | A | |
| 31 | SEQ ID NO:19 | FLL | R | Y | I | |
| 32 | SEQ ID NO:20 | FLL | S | V | I | |
| 33 | | (F)LL | A | T | I | HAV |
| 34 | | (F)LL | D | S | Q | (Whole |
| 35 | | (F)LL | E | I | K | Blood) |
| 36 | | (F)LL | H | P | I | |
| 37 | SEQ ID NO:21 | FLL | F | R | H | |
| 38 | SEQ ID NO:22 | FLL | K | D | Q | |
| 39 | SEQ ID NO:23 | FLL | Q | Y | K | |
| 40 | SEQ ID NO:24 | FLL | T | G | K | |
| 41 | SEQ ID NO:25 | FLL | Y | F | T | |
| 42 | | (F)LL | V | L | P | |
| 43 | | (F)LL | T | S | Y | ALL 3 |
| 44 | SEQ ID NO:26 | FLL | D | L | X | Viruses |
| 45 | | (F)LL | D | K | X | |
| 46 | SEQ ID NO:27 | FLL | Y | A | K | |

The frequency of the different amino acids in positions 4-6 is summarized in Table 5. As stated above, X represents an amino acid whose identity remained ambiguous after sequencing, and may be any of the amino acids used in the construction of the library.

Three amino acids frequently detected in positions 4-6 are P, Y, and K, respectively. The sequences FLLPYK (SEQ ID NO: 28) and (F)LLHPI are selected as consensus peptides to be used for further research in the removal of virus from blood samples.

TABLE 5

| # | A | D | E | F | G | H | I | K | L | N | P | Q | R | S | T | V | Y |
|---|---|---|---|---|---|---|---|---|---|---|---|---|---|---|---|---|---|
| 4 | 4 | 4 | 1 | 2 | 0 | 5 | 0 | 4 | 1 | 0 | 5 | 5 | 5 | 1 | 2 | 1 | 4 |
| 5 | 3 | 1 | 2 | 6 | 4 | 2 | 2 | 2 | 3 | 1 | 5 | 1 | 3 | 3 | 1 | 1 | 4 |
| 6 | 1 | 0 | 1 | 3 | 0 | 2 | 5 | 10 | 2 | 0 | 2 | 2 | 2 | 2 | 3 | 1 | 6 |
| 4-6 | 8 | 5 | 4 | 11 | 4 | 9 | 7 | 16 | 6 | 1 | 12 | 8 | 10 | 6 | 6 | 3 | 14 |

Peptides are synthesized directly onto Toyopearl 650 amino resin, or onto a larger pore resin from TosoHaas (e.g., Toyopearl 750 amino). The first experiment evaluates whether PPV bound to the peptide FLLPYK (SEQ ID NO: 28). Saline is spiked with PPV, and the solution is passed through a column of FLLPYK synthesized onto amino resin. An epsilon amino acid is included at the carboxy terminal of the peptide to act as a spacer between the peptide and the resin. Samples of the starting material and flow-through are assayed for infectious PPV. Typically five logs reduction of virus are found in the flow through of the column with the FLLPYK (SEQ ID NO: 28) peptide, while 3 logs of removal are found with amino resin alone. A one log reduction means 90% removal, two logs means 99%, etc. The contact time is four minutes and the experiment is performed at room temperature. Amino-FLLPY (SEQ ID NO: 3 1) gives 4.5 logs of clearance, while amino-FLLP (SEQ ID NO: 32) and amino-FLL give no significant clearance. Acetyl-FLLPYK (SEQ ID NO: 28) gives no clearance, nor does acetyl-FLLPY (SEQ ID NO: 3 1). Thus, the terminal amino group of this peptide is necessary for efficient binding to PPV. Comparable experiments show that amino-FLLPYK (SEQ ID NO: 28) binds HAV, PRV, HIV and BVDV.

A series of experiments with the sequence (F)LLHPI are conducted with both free amino- and acetylated amino termini. Saline spiked with PPV (10 mL) is passed over a resin (1 mL) containing the sequence (F)LLHPI with a residence time of 4 minutes. This column provides 5 logs of clearance. In identical experiments with amino resin, about 2-3 logs of clearance are found. This suggests that two methods of virus removal are occurring: 1) affinity interaction with the peptide sequence and 2) weaker ion exchange with the free amino group. When the free amino groups are acetylated, the base resin (acetylated amino resin) clears little virus (less than 1 log), while acetylated-(F)LLHPI binds 2-3 logs of PPV.

The ability of the sequence acetylated-(F)LLHPI to selectively bind virus is further demonstrated by the addition of PPV to plasma. Ten mL of PPV spiked plasma is passed through 1 ml of resin. A total of two logs of PPV are cleared. When the plasma is passed over the same resin again, 3.2 logs are removed. When recycled, 3.5 logs are removed and when passed for a third time, a total of 4.7 logs are cleared from plasma. Thus, virus can be substantially bound to the resin, i.e. more than 99.99% removed from plasma. This resin is also shown to bind PRV, HAV, HIV, EMCV, BVDV, B19 and SV40.

### EXAMPLE 4

Identification of peptides that bind to prions using QuASAR™ screening

The QUASAR™ screening method is also used to identify ligands specific for prion particles. Peptide libraries are regenerated and pre-screened with monoclonal antibody FH11 (provided by Jim Hope of the Animal Health Institute, Compton, Berkshire, UK; see also Foster et al., Vet. Rec. 139:512-515, (1996)), which was raised to the amino terminal region of a recombinant PrP protein. The ligand library is then regenerated and probed with brain extract from normal hamsters that were litter mates of animals infected with scrapie particles. Four ligands for normal prion are identified as indicated in Table 6.

Following regeneration, the library is screened for ligands that bind to extracts of brain from hamsters infected with scrapie. An additional ten ligands are identified as indicated in Table 6. A total of 14 different ligands specific for scrapie protein are identified, 12 of which are sequenced. The sequencing results are given in Table 6.

TABLE 6

| | | | | | | | | Normal | Infected |
|---|---|---|---|---|---|---|---|---|---|
| Sample | SEQ ID | 1 | 2 | 3 | 4 | 5 | 6 | prion | prion |
| 1 | | (R) | A | A | T | E | H | | + |
| 2 | | (H) | H | H | P | Q | T | | + |
| 3 | | (V) | S | H | L | L | S | | + |
| 4 | | (T) | L | H | E | T | L | | + |
| 5 | | (V) | A | G | Q | G | Q | | + |
| 6 | | (S) | D | F | L | K | R | | + |
| 7 | | (V) | F | V | R | F | X | | + |
| 8 | | (V) | A | K | V | S | P | + | + |
| 9 | | (R) | Y | H | V | Y | F | + | + |
| 10 | | (E) | R | P | D | K | G | + | + |
| 11 | SEQ ID NO:29 | Y | R | N | Q | F | R | | + |
| 12 | SEQ ID NO:30 | A | V | F | N | F | D | + | + |

The foregoing description has been presented only for the purposes of illustration and is no intended to limit the invention to the precise form disclosed, but by the claims appended hereto.

What is claimed is:

1. A peptide less than 20 amino acids in length and comprising a sequence selected from the group consisting of a PPV-binding domain, an HAV-binding domain, and a prion-binding domain.

2. The peptide of claim 1, wherein said peptide comprises an HAV-binding domain.

3. The peptide of claim 2, wherein said HAV-binding domain binds to HAV in the presence of fibrinogen.

4. The peptide of claim 3, wherein said HAV-binding domain comprises a sequence selected from the group consisting of: FLLFRF (SEQ ID NO: 9); FLLHEE (SEQ ID NO: 10); FLLHPH (SEQ ID NO: 11); FLLHSL (SEQ ID NO: 12); FLLRKF (SEQ ID NO: 13); FLLRYS (SEQ ID NO: 14); FLLYRY (SEQ ID NO: 15); (F)LLDIR; (F)LLKFP; (F)LLKQI; (F)LLPLK; (F)LLQAY; (F)LLQHY; (F)LLRFT; (F)LLYGK; (F)LLATI; (F)LLDSQ; (F)LLEIK; (F)LLHPI; FLLFRH (SEQ ID NO: 21); FLLKDQ (SEQ ID NO: 22); FLLQYK (SEQ ID NO: 23); FLLTGK (SEQ ID NO: 24); FLLYFT (SEQ ID NO: 25) and (F)LLVLP.

5. The peptide of claim 1, wherein said peptide comprises a PPV-binding domain.

6. The peptide of claim 5, wherein said PPV-binding domain binds to PPV in the presence of fibrinogen.

7. The peptide of claim 6, wherein said PPV-binding domain comprises a sequence selected from the group consisting of (F)LLAEY; (F)LLAFS; (F)LLAGV; (F)LLHHI; (F)LLKGY; (F)LLLPK; (F)LLPAK; (F)LLPFL; (F)LLPPR; (F)LLPYK; FLLQNK (SEQ ID NO: 16); FLLQPF (SEQ ID NO: 17); FLLRFA (SEQ ID NO: 18); FLLRYT (SEQ ID NO: 19); and FLLSVI (SEQ ID NO: 20).

8. The peptide of claim 1, wherein said peptide comprises a HAV-binding domain.

9. The peptide of claim 8, wherein said HAV-binding domain binds to HAV in the presence of fibrinogen.

10. The peptide of claim 9, wherein said HAV-binding domain comprises a sequence selected from the group consisting of FLLPYK (SEQ ID NO: 28); (F)LLHPI; (F)LLTSY; FLLDLX (SEQ ID NO: 26); (F)LLDKX; and FLLYAK (SEQ ID NO: 27).

11. The peptide of claim 2, wherein said HAV-binding domain binds PRV.

12. The peptide of claim 11, wherein said HAV-binding domain comprises a sequence selected from the group consisting of FHALRH (SEQ ID NO: 1); FFSKQN (SEQ ID NO: 2); (F)AAFIN; (F)LLTSY; (F)LKLFP; (F)PNGGI; (F)VEVKF; FPLIKA (SEQ ID NO: 3); (F)FFTFK; (F)LLDLX; (F)YYLNV; FLILDP (SEQ ID NO: 4); FYTPPY (SEQ ID NO: 5); FFYPAX (SEQ ID NO: 6); FLLDKX (SEQ ID NO: 7) and FLLYAK (SEQ ID NO: 8).

13. The peptide of claim 1, wherein said peptide comprises a sequence which binds to a prion protein.

14. The peptide of claim 13, wherein said prion protein-binding sequence is selected from the group consisting of (R)AATEH; (H)HHPQT; (V)SHLLS; (T)LHETL; (V)AGQGQ; (S)DFLKR; (V)FVRFX; (V)AKVSP; (R)YHVYF; (E)RPDKG; YRNQFR (SEQ ID NO: 29); and AVFNFD (SEQ ID NO: 30).

15. The peptide of claim 1, wherein said peptide is less than 15 amino acids in length.

16. The peptide of claim 1, wherein said peptide is less then 10 amino acids in length.

17. The peptide of claim 1, wherein said peptide consists of the amino acid sequence selected from the group consisting of FHALRH (SEQ ID NO: 1); FFSKQN (SEQ ID NO: 2); (F)AAFIN; (F)LLTSY; (F)LKLFP; (F)PNGGI; (F)VEVKF; FPLIKA (SEQ ID NO: 3); (F)FFTFK; (F)LLDLX; (F)YYLNV; FLILDP (SEQ ID NO: 4); FYTPPY (SEQ ID NO: 5); FFYPAX (SEQ ID NO: 6); FLLDKX (SEQ ID NO: 7); FLLYAK (SEQ ID NO: 8); FLLPYK (SEQ ID NO: 28); (F)LLHPI; (R)AATEH; (H)HHPQT; (V)SHLLS; (T)LHETL; (V)AGQGQ; (S)DFLKR; (V)FVRFX; (V)AKVSP; (R)YHVYF; (E)RPDKG; YRNQFR (SEQ ID NO: 29); AVFNFD (SEQ ID NO: 30); FLLFRF (SEQ ID NO: 9); FLLHEE (SEQ ID NO: 10); FLLHPH (SEQ ID NO: 11); FLLHSL (SEQ ID NO: 12); FLLRKF (SEQ ID NO: 13); FLLRYS (SEQ ID NO: 14); FLLYRY (SEQ ID NO: 15); (F)LLDIR; (F)LLKFP; (F)LLKQI; (F)LLPLK; (F)LLQAY; (F)LLQHY; (F)LLRFT; (F)LLYGK; (F)LLATI; (F)LLDSQ; (F)LLEIK; (F)LLHPI; FLLFRH (SEQ ID NO: 21); FLLKDQ (SEQ ID NO: 22); FLLQYK (SEQ ID NO: 23); FLLTGK (SEQ ID NO: 24); FLLYFT (SEQ ID NO: 25); (F)LLVLP; (F)LLAEY; (F)LLAFS; (F)LLAGV; (F)LLHHI; (F)LLKGY; (F)LLLPK; (F)LLPAK; (F)LLPFL; (F)LLPPR; (F)LLPYK; FLLQNK (SEQ ID NO: 16); FLLQPF (SEQ ID NO: 17); FLLRFA (SEQ ID NO: 18); FLLRYT (SEQ ID NO: 19); FLLSVI (SEQ ID NO: 20); (F)LLTSY; FLLDLX (SEQ ID NO: 26); (F)LLDKX; and FLLYAK (SEQ ID NO: 27).

18. A composition comprising the peptide of claim 1.

19. The composition of claim 18, wherein said peptide is coupled to a solid support.

20. The composition of claim 19, wherein said solid support is a resin.

21. A method of removing a target from a biological fluid, the method comprising contacting the biological fluid with the composition of claim 18 under conditions sufficient to cause specific binding of said target to said peptide in said composition.

22. The method of claim 21, wherein said target is selected from the group consisting of HAV, PPV, PRV, prion protein, HIV, EMCV, BVDV, B19 and SV40.

23. The method of claim 21, wherein said biological fluid is selected from the group consisting of blood, plasma, serum, cerebrospinal fluid, urine, saliva, milk, ductal fluid, tears, and semen.

24. A peptide less than 20 amino acids in length and comprising a sequence selected from the group consisting of an EMCV-binding domain, an SV40-binding domain, a poliovirus-binding domain, a BVDV-binding domain, and an API-binding domain.

25. The peptide of claim 24, wherein said peptide comprises an EMCV-binding domain.

26. The peptide of claim 25, wherein said EMCV-binding domain comprises a sequence selected from the group consisting of FLLRNV (SEQ ID NO: 33), FLLNAH (SEQ ID NO: 34), FLLGPR (SEQ ID NO: 35), and FLLNQE (SEQ ID NO: 36).

27. The peptide of claim 24, wherein said peptide comprises an SV40-binding domain.

28. The peptide of claim 27, wherein said SV40-binding domain comprises a sequence selected from the group consisting of FLLFQP (SEQ ID NO: 37), FLLEVY (SEQ ID NO: 38), and FLLRGS (SEQ ID NO: 39).

29. The peptide of claim 24, wherein said peptide comprises a poliovirus-binding domain.

30. The peptide of claim 29, wherein said poliovirus-binding domain comprises a sequence selected from the group consisting of FLLIDA (SEQ ID NO: 40), FLLQSA (SEQ ID NO: 41), FLLKEI (SEQ ID NO: 42), FLLPFK (SEQ ID NO: 43), FLLAPN (SEQ ID NO: 44), FLLYSA (SEQ ID NO: 45), FLLLNS (SEQ ID NO: 46), FLLYRR (SEQ ID NO: 47), and FLLKSV (SEQ ID NO: 48).

31. The peptide of claim 24, wherein said peptide comprises a BVDV-binding domain.

32. The peptide of claim 31, wherein said BVDV-binding domain comprises a sequence selected from the group consisting of FLLLRN (SEQ ID NO: 49), and FLLRGH (SEQ ID NO: 50).

33. The peptide of claim 24, wherein said peptide comprises an API-binding domain.

34. The peptide of claim 33, wherein said API-binding domain comprises a sequence selected from the group consisting of AQTFHD (SEQ ID NO: 51), RDYDTD (SEQ ID NO: 52), LKRIEY (SEQ ID NO: 53), SDLRRL (SEQ ID NO: 54), APPRTV (SEQ ID NO: 55), VLYTNN (SEQ ID NO: 56), NFZQNT (SEQ ID NO: 57), and SKNNAA (SEQ ID NO: 58).

35. A composition comprising the peptide of claim 24.

36. The composition of claim 35, wherein said peptide is coupled to a solid support.

37. The composition of claim 36, wherein said solid support is a resin.

38. A method of removing a target from a biological fluid, the method comprising contacting the biological fluid with the composition of claim 35 under conditions sufficient to cause specific binding of said target to said peptide in said composition.

39. The method of claim 38, wherein said target is selected from the group consisting of EMCV, SV40, BVDV, API, and poliovirus.

40. The method of claim 38, wherein said biological fluid is selected from the group consisting of blood, plasma, serum, cerebrospinal fluid, urine, saliva, milk, ductal fluid, tears, and semen.

41. A method for identifying a ligand for a target, the method comprising:
a) incubating a plurality of immobilized ligands with a first solution substantially free of said target and comprising one or more agents under conditions which allow for formation of stable complexes between said ligands and agents;
b) contacting said ligand-agent complexes with a probe molecule having an affinity for said target;
c) identifying probe molecules bound to said ligand-agent complexes by identifying a transient signal associated with said complexes;
d) incubating said ligands with a second solution comprising said target and said first solution under conditions allowing for formation of stable complexes between said target and ligands and for formation of stable complexes between said agents and said ligands;
e) contacting said ligand-target complexes and ligand-agent complexes with said probe molecule;
f) identifying probe molecules bound to ligand-target complexes and probe molecules bound to said ligand-agent complexes in said second solution by detecting said transient signal, and
g) comparing the transient signal associated with ligand-probe complexes in said second solution with the transient signal associated with ligand-probe complexes in said first solution,
wherein a signal preferentially associated with said second solution to said first solution indicates a ligand-probe molecule complex comprising a ligand specific for said target.

42. The method of claim 41, wherein the ligands are immobilized on individual supports, thereby creating a population of supports.

43. The method of claim 42, wherein the individual supports are beads.

44. The method of claim 41, wherein the plurality of immobilized ligands comprises a combinatorial library of ligand-containing beads.

45. The method of claim 42, wherein the population of supports is immobilized on a surface.

46. The method of claim 45, wherein the surface is planar.

47. The method of claim 46, wherein the surface is transparent.

48. The method of claim 42, wherein the population of supports is embedded in an adhesive.

49. The method of claim 48, wherein the adhesive is an elastomeric sealant.

50. The method of claim 49, wherein the adhesive is a vinyl adhesive caulking material.

51. The method of claim 41, wherein the ligand molecules are polymeric.

52. The method of claim 41, wherein the ligand molecules are selected from the group consisting of peptides, peptidomimetics, small organic molecules, nucleic acids, and carbohydrates.

53. The method of claim 41, wherein the probe molecule is an antibody.

54. The method of claim 41, wherein the transient signal is chemiluminescence.

55. The method of claim 54, wherein the chemiluminescence is detected by autoradiography.

56. The method of claim 41, wherein the target is a cell.

57. The method of claim 41, wherein the target is a virus.

58. The method of claim 57, wherein the virus is porcine parvovirus.

59. The method of claim 57, wherein the virus is pseudorabies virus.

60. The method of claim 57, wherein the virus is hepatitis A virus.

61. The method of claim 57, wherein the virus is poliovirus.

62. The method of claim 57, wherein the virus is simian virus 40.

63. The method of claim 57, wherein the virus is encephalomyocarditis virus.

64. The method of claim 57, wherein the virus is bovine viral diarrhea virus.

65. The method of claim 41, wherein the target is a protein.

66. The method of claim 65, wherein the protein is alpha-1-proteinase inhibitor.

67. The method of claim 65, wherein the protein is a prion.

68. The method of claim 67, wherein the prion is scrapie prion.

69. The method of claim 41, further comprising removing the agent from said ligand-agent complexes after detecting said transient signal associated with said complexes, and before incubating said ligands with said second solution.

70. The method of claim 41, further comprising determining the identity of ligands specific for said target.

71. A method for identifying a ligand for a plurality of targets, the method comprising:
(a) incubating a plurality of immobilized ligands with a first solution substantially free of a first target and comprising one or more agents under conditions which allow for formation of stable complexes between said ligands and agents;
(b) contacting said ligand-agent complexes with a probe molecule having an affinity for said first target;
(c) identifying probe molecules bound to said ligand-agent complexes by identifying a transient signal associated with said complexes;
(d) removing said agent from said ligand-agent complexes;
(e) incubating said ligands with a second solution comprising said first target and said first solution under conditions allowing for formation of stable complexes between said first target and ligands and for formation of stable complexes between said agents and said ligands;
(f) contacting said ligand-first target complexes and ligand-agent complexes with said probe molecule;
(g) identifying probe molecules bound to ligand-first target complexes and probe molecules bound to said ligand-agent complexes in said second solution by detecting said transient signal;
(h) comparing the transient signal associated with ligand-probe complexes in said second solution with the transient signal associated with ligand-probe complexes in said first solution, wherein a signal preferentially associated with said second solution to said first solution indicates a ligand-probe molecule complex comprising a ligand specific for said first target;
(i) removing said agents from said ligand-agent complexes and said first targets from said ligand-first target complexes, and
j) repeating steps (e) through (i) with a third solution comprising a second target.

72. The method of claim 71, further comprising determining the identity of a ligand specific for first target or second target.

73. A method for identifying a peptide that binds to a virus present in a blood composition, the method comprising:
(a) providing a plurality of immobilized beads comprising a combinatorial peptide library;
(b) incubating said peptide library with a blood composition substantially free of said virus and comprising one or more agents, under conditions which allow for formation of stable complexes between said peptides and agents;
(c) contacting said complexes with a first antibody having an affinity for said virus under conditions that allow formation of peptide-agent-first antibody complexes;
(d) identifying a transient signal associated with said peptide-agent complexes;
(e) contacting said peptides with a second blood composition comprising said virus molecule and said first blood composition under conditions allowing for formation of stable peptide-virus complexes and peptide-agent complexes;
(g) identifying a transient signal associated with said peptide-virus complexes and peptide-agent complexes; and
(h) comparing said transient signal associated with peptide-virus complexes and said transient signal associated with peptide-agent complexes with said transient signal associated with peptide-agent complexes in said first blood composition, wherein a transient signal preferentially associated with said second blood composition to said first blood composition indicates a peptide-virus complex comprising a peptide specific for said virus.

74. The method of claim 73, further comprising determining the identity of a ligand specific for first target or second target.

75. The method of claim 73, further comprising identifying the sequences of said peptides.

76. The method of claim 73, wherein said peptide library is immobilized on a transparent film using a vinyl caulking adhesive.

77. The method of claim 73, further comprising repeating steps (e) through (i) with a third blood composition comprising a second target.

78. The method of claim 77, further comprising repeating steps (e) through (i) with a fourth blood composition comprising a third target.
